(19) United States
(12) Patent Application Publication
(10) Pub. No.: US 2010/0087738 A1
(43) Pub. Date: Apr. 8, 2010

(54) SYSTEMS AND METHODS FOR QUANTIFYING CARDIAC DYSSYNCHRONY

(76) Inventors: Brandon K. Fornwalt, Decatur, GA (US); John N. Oshinski, Decatur, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/440,825

(22) PCT Filed: Sep. 11, 2007
(86) PCT No.: PCT/US07/78112

§ 371 Date: Oct. 26, 2009

Related U.S. Application Data
(60) Provisional application No. 60/843,633, filed on Sep. 11, 2006.
(60) Provisional application No. 60/846,240, filed on Oct. 13, 2006.

(51) Int. Cl. A61B 8/13; G06K 9/00

(52) U.S. Cl. 600/453; 382/128

(57) ABSTRACT

In one embodiment, a system and method for quantifying cardiac dyssynchrony relate to capturing images of the heart over time as it beats, generating heart function profiles for discrete portions of the myocardium from the captured images, the heart function profiles each pertaining to a heart function parameter as a function of time, and performing cross-correlation on the generated heart function profiles to yield a cross-correlation function that identifies a time delay at which the heart function profiles temporally correlate most closely, the time delay being indicative of a degree of cardiac dyssynchrony.

### CROSS-REFERENCE TO RELATED APPLICATION

This application claims priority to copending U.S. provisional applications entitled, “Quantifying the Synchrony of Contraction and Relaxation in the Heart”, having Ser. No. 60/843,633, filed Sep. 11, 2006 and “Systems And Methods For Quantifying Cardiac Dyssynchrony”, having Ser. No. 60/846,240, filed Oct. 13, 2006, both of which are entirely incorporated herein by reference.

### BACKGROUND

Cardiac resynchronization therapy (CRT) has been found to be effective in treating many patients with severe cardiac disease. In such therapy, a “pacemaker” is used to contract the myocardium at regular periodic intervals to control the pumping of blood to the rest of the body. One reason that CRT is believed to be effective is that it, at least partially, overcomes cardiac dyssynchrony, a phenomenon in which different parts of the heart contract temporally out of phase. Such dyssynchrony is believed to be both a chronic problem, because it causes enlargement of the heart, and an acute problem, because it reduces the efficiency with which the heart pumps blood.

Given that CRT can be beneficial to heart patients suffering from dyssynchrony, it is important to be able to identify those patients who exhibit such dyssynchrony. At one time, dyssynchrony was diagnosed by performing an electrocardiogram (EKG). It has been found, however, that the EKGs only provide somewhat anecdotal evidence as to the presence of dyssynchrony. Therefore, more recently, tissue Doppler velocity imaging (TDI) has been used to determine the relative velocities of discrete points of the heart to identify any dyssynchrony that exists between those parts. For example, TDI may be used to develop a velocity curve for a first point on the left ventricular wall and for a second point on the left ventricular wall opposite the first point. Once the velocity curves are developed, the peak velocities of the curves during the systolic phase of the cardiac cycle are compared and the time difference between the two peaks is used as a measure of dyssynchrony.

Although current dyssynchrony diagnosis using TDI is more effective than previous diagnosis performed relative to EKG data, the current techniques are somewhat limited given that the results obtained can be inconsistent. In particular, the time differential between systolic velocity peaks observed from one area of the heart may indicate relatively high dyssynchrony, while the time differential between peaks observed from another area of the heart may indicate relatively low dyssynchrony. One reason for such incongruent results may be due to the fact that the dyssynchrony diagnosis is only made in relation to the peak systolic velocity, i.e., a single point on each velocity curve. By only considering a single point of each curve, the effects of noise are more significant and may skew the results of the analysis.

### BRIEF DESCRIPTION OF THE FIGURES

The components in the drawings are not necessarily to scale, emphasis instead being placed upon clearly illustrating the principles of the present disclosure. In the drawings, like reference numerals designate corresponding parts throughout the several views.

FIG. 1 is a block diagram of an embodiment of a system for collecting data pertaining to heart function and for quantifying dyssynchrony relative to the collected data.

FIG. 2 is a block diagram of an embodiment of a computer shown in FIG. 1.

FIG. 3 is a flow diagram of an embodiment of a method for quantifying cardiac dyssynchrony.

FIG. 4 is a graph showing velocity curves for two discrete locations of a patient's heart.

FIG. 5 is a graph showing a cross-correlation function that quantifies dyssynchrony relative to the velocity curves of FIG. 4.

FIG. 6 is a graph showing the velocity curves of FIG. 4 after one of the curves has been shifted in time relative to the dyssynchrony quantified by the cross-correlation function of FIG. 5.

FIG. 7 comprises graphs showing velocity curves and a cross-correlation function for a representative negative control subject.

FIG. 8 comprises graphs showing velocity curves and cross-correlation functions for a representative positive control before and after CRT.

FIG. 9 comprises graphs showing values of dyssynchrony parameters for all positive control subjects before and after CRT.

FIG. 10 comprises graphs showing values of the dyssynchrony parameters for all control group subjects along with threshold values used to diagnose dyssynchrony.

FIG. 11 is a graph showing a receiver operating characteristic comparison of dyssynchrony parameters.

### DETAILED DESCRIPTION

As described above, current dyssynchrony diagnosis techniques are somewhat limited given that they only consider dyssynchrony at the peak ventricular wall velocity during systole. As described below, disclosed are systems and methods with which dyssynchrony is quantified relative to a period of the cardiac cycle rather than a discrete point of that cycle. Under such a scheme, the effects of noise may be reduced and more accurate quantification of dyssynchrony may be obtained. In some embodiments, the period that is considered comprises one or more complete cardiac cycles. In other embodiments, one or more portions of the cardiac cycle, such one or more systolic phases or one or more diastolic phases, are considered. As described in greater detail in the following, cross-correlation of profiles that pertain to the periods of interest can be performed to determine a time value indicative of any dyssynchrony that is present. In some embodiments, that time value can then be compared to empirical data taken from test subjects to make a determination as to whether a patient is or is not a good candidate for CRT.

Described in the following are various embodiments of systems and methods for quantifying cardiac dyssynchrony. Although particular embodiments are described, those embodiments are mere example implementations of the systems and methods and it is noted that other embodiments are possible. All such embodiments are intended to fall within the scope of this disclosure.

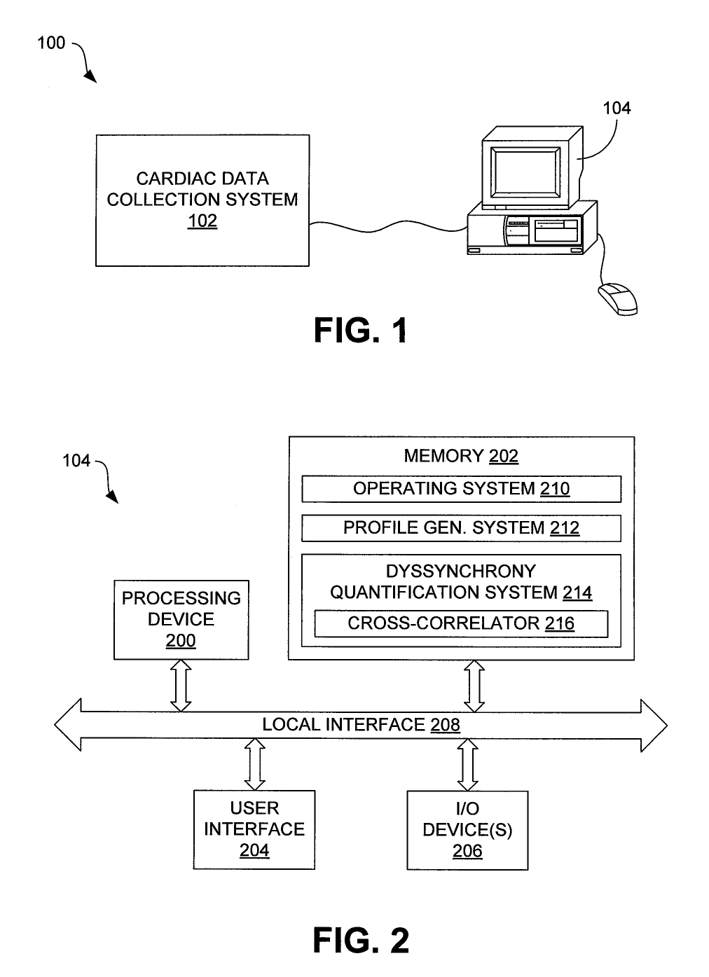

FIG. 1 illustrates an example system 100 with which cardiac function can be evaluated. As indicated in FIG. 1, the system 100 generally comprises a cardiac data collection system 102 and a computer 104 that are coupled such that data can be sent from the data collection system to the computer. By way of example, the system 100 comprises part of a network, such as a local area network (LAN) or wide area network (WAN).

As its name suggests, the cardiac data collection system 102 is configured to collect data as to functioning of the heart. More particularly, the data collection system 102 is configured to collect data relating to contraction of the myocardium as the heart beats. In some embodiments, the data collection system comprises an imaging system that is configured to capture images of the heart over time as a means of identifying motion of discrete portions of the myocardium during the cardiac cycle. Such an imaging system can comprise substantially any form of imaging system with which relatively high resolution images can be captured. Examples include tissue Doppler velocity imaging (TDI) systems, magnetic resonance imaging (MRI) systems, and computed tomography (CT) imaging systems.

As described below, the computer 104, and more particularly software provided on the computer, is configured to receive the data collected by the cardiac data collection system 102 and evaluate that data to quantify cardiac dyssynchrony. Notably, although the data collection system 102 and the computer 104 are illustrated as separate components in FIG. 1, the two components and/or one or more of their respective functionalities can be integrated into a single system or machine, if desired.

FIG. 2 is a block diagram illustrating an example architecture for the computer 104 shown in FIG. 1. The computer 104 of FIG. 2 comprises a processing device 200, memory 202, a user interface 204, and at least one I/O device 206, each of which is connected to a local interface 208.

The processing device 200 can include a central processing unit (CPU) or a semiconductor-based microprocessor in the form of a microchip. The memory 202 includes any one of a combination of volatile memory elements (e.g., RAM) and nonvolatile memory elements (e.g., hard disk, ROM, tape, etc.).

The user interface 204 comprises the components with which a user interacts with the computer 104 and therefore may comprise, for example, a keyboard, mouse, and a display, such as a liquid crystal display (LCD) monitor. The one or more I/O devices 206 are adapted to facilitate communications with other devices or systems and may include one or more communication components such as a modulator/demodulator (e.g., modem), wireless (e.g., radio frequency (RF)) transceiver, network card, etc.

The memory 202 comprises various software programs including an operating system 210, cardiac profile generation system 212, and a dyssynchrony quantification system 214. The operating system 210 controls the execution of other programs and provides scheduling, input-output control, file and data management, memory management, and communication control and related services. The cardiac profile generation system 212 generates heart function profiles or curves that relate to functioning of the heart over time. By way of example, the cardiac profile generation system 212 generates velocity profiles, displacement profiles, strain rate profiles, or strain profiles from the data collected by the cardiac data collection system 102. In some embodiments, the profiles are generated relative to data pertaining to discrete portions of the walls of a heart ventricle, such as a left ventricle.

The dyssynchrony quantification system 214 is configured to receive the function profiles generated by the cardiac profile generation system 212. In the embodiment of FIG. 2, the dyssynchrony quantification system 214 comprises a cross-correlator 216 that is configured to cross-correlate the received profiles to determine time delays between the profiles, those time delays being indicative of dyssynchrony. In some embodiments, the cross-correlator 216 generates a cross-correlation function whose maximum identifies the time delay. Although a cross-correlation has been specifically described, it is to be understood that other mathematical techniques could be used to quantify dyssynchrony using the collected data. Examples of such other techniques include: Granger causality, directed transfer function, direct directed transfer function, short-time directed transfer function, bivariate coherence, and partial directed coherence.

Various programs (i.e. logic) have been described herein. Those programs can be stored on any computer-readable medium for use by or in connection with any computer-related system or method. In the context of this document, a computer-readable medium is an electronic, magnetic, optical, or other physical device or means that contains or stores a computer program for use by or in connection with a computer-related system or method. Those programs can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

Example systems having been described above, operation of the systems will now be discussed. In the discussion that follows, a flow diagram is provided. It is noted that process steps or blocks in that flow diagram may represent modules, segments, or portions of code that include one or more executable instructions for implementing specific logical functions or steps in the process. Although particular example process steps are described, alternative implementations are feasible. Moreover, steps may be executed out of order from that shown or discussed, including substantially concurrently or in reverse order, depending on the functionality involved.

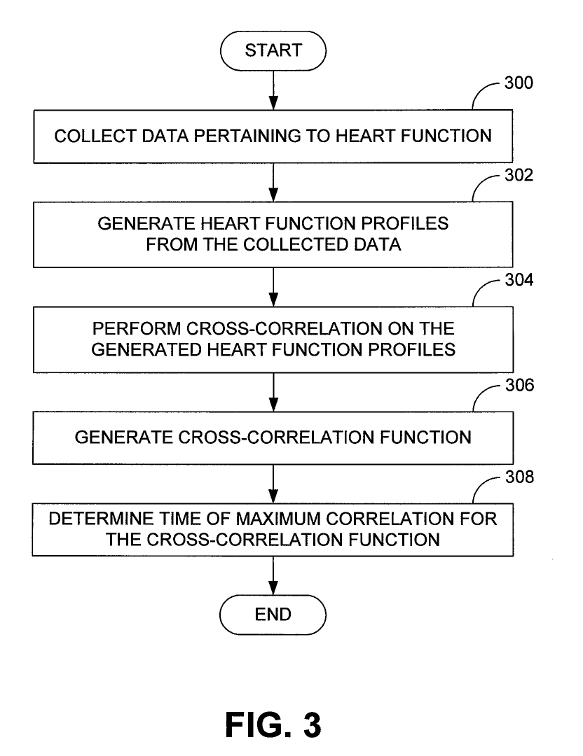

FIG. 3 illustrates an embodiment of a method for quantifying dyssynchrony. Beginning with block 300, data pertaining to heart function is collected, for example by the cardiac data collection system 102 (FIG. 1). By way of example, the data comprises images of the heart that are captured over time during a predetermined portion of the cardiac cycle, such as one or more complete cardiac cycles, one or more systole phases, or one or more diastole phases. In some embodiments, images are captured from one or more of apical 2-, 3-, and 4-chamber views.

Next, with reference to block 302, heart function profiles are generated from the collected data, for example by the cardiac profile generation system 212 (FIG. 2). In some embodiments, the profiles are generated in relation to pairs of discrete portions of the myocardial walls, for example pairs of points located on opposite walls of the left ventricle, whose function may be most indicative of life-threatening dyssynchrony. By way of example, the profiles are generated relative to the basal segments of the left ventricle walls.

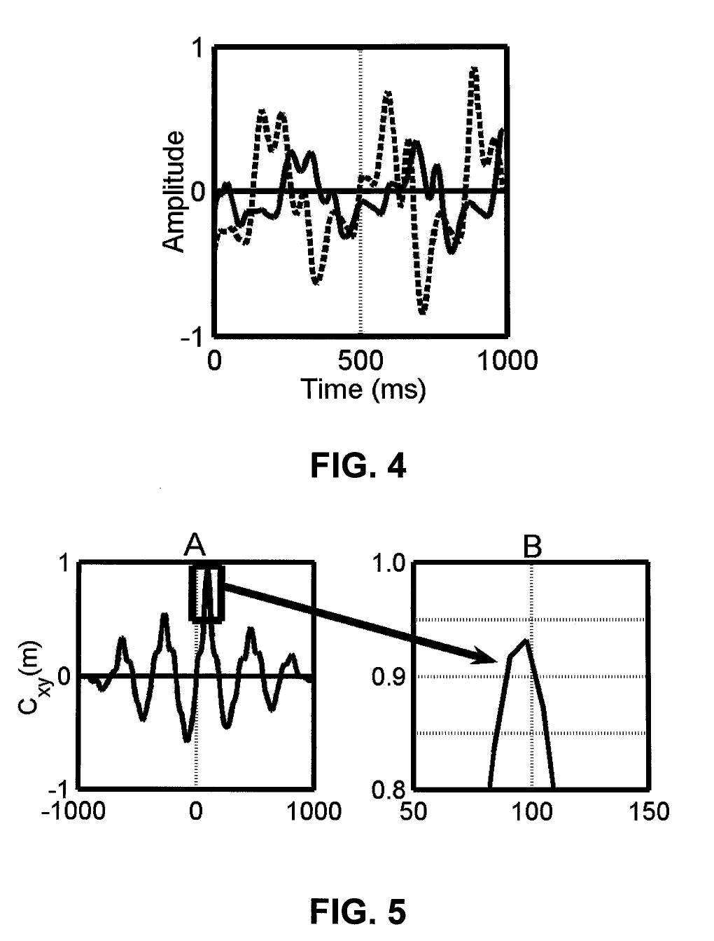

In some embodiments, the profiles comprise velocity profiles or curves that identify the velocity of the myocardial portions of interest versus time. An example of such velocity curves is provided in FIG. 4. As shown in that figure, two velocity curves are plotted for a period of 1000 milliseconds (ms), each curve pertaining to one of two opposed points on the walls of the myocardium (e.g., left ventricle walls). As is apparent from FIG. 4, the two curves are similar functions of time but are out of phase due to cardiac dyssynchrony.

In other embodiments, the profiles are related to another heart function parameter. For example, the profiles can comprise displacement profiles or curves that identify the positions of the discrete portions of the myocardium as functions of time. Such curves can be generated by performing integration of the velocity curves, which may remove noise associated with the data capture process. As another example, the profiles can comprise strain rate profiles or curves that identify strain rate of the discrete portions of the myocardium as functions of time. Such curves can be generated through comparison of the velocities of adjacent portions of the myocardial walls to estimate the stain rate applicable to those walls. As a further example, the profiles can comprise strain profiles or curves that identify strain of the discrete portions of the myocardium as functions of time. Such curves can be generated by performing integration of the strain rate curves.

Irrespective of the particular parameter to which the heart function profiles pertain, cross-correlation is then performed on the profiles, as indicated in block 304, for example by the dyssynchrony quantification system 214 and, more particularly, by the cross-correlator 216 (FIG. 2). In such a process, a cross-correlation function C_{xy}(m) is calculated as between the pairs of heart function profiles for various temporal delays, m, according to the following relation:

$${C}_{xy} ( m ) = \{ \begin{matrix}{\sum }_{n = o}^{N - m - 1} {x}_{n + m} * {y}_{n} & for all m \geq 0 \\ {C}_{yx} ( - m ) & for all m < 0\end{matrix} \qquad [ Equation 1 ]$$

where x and y are myocardial parameter (e.g., velocity) vectors of the two heart function profiles, N is the number of data points, and m is an integer. Using that relation, a cross-correlation for each value of m is calculated, and a cross-correlation function can be generated, as indicated in block 306. FIG. 5 illustrates an example cross-correlation function (see part A of FIG. 5) that results from cross-correlation of the two velocity curves shown in FIG. 4. The cross-correlation function is formed from a plurality of data points, each data point representing the level of correlation between the two velocity curves at a different value of m. Therefore, in essence, the cross-correlation function is generated by comparing the curves at a multiplicity of time delays to identify the time delay at which the two curves correlate most closely.

Referring next to block 308 of FIG. 3, the time of maximum correlation is determined for the cross-correlation function. That action can be performed by simply identifying the maximum value of the cross-correlation function. In the example of FIG. 5, that maximum occurs at approximately 98 milliseconds (ms) (see part B of FIG. 5). Therefore, the two portions of the myocardium represented by the two velocity curves of FIG. 4 are out of synchronization, or dyssynchronous, by approximately 98 ms.

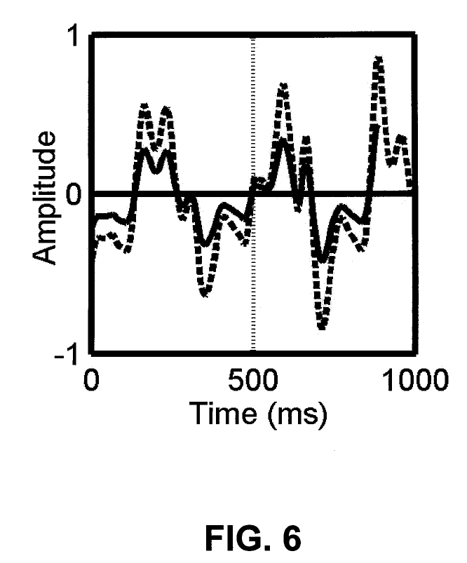

FIG. 6 shows the velocity curves of FIG. 4 after one of the curves (i.e., the solid line curve) has been shifted in time in an amount equal to the time of maximum correlation, i.e., 98 ms, the time delay quantification of the dyssynchrony. As is apparent from FIG. 6, the curves are in substantial temporal registration with each other after such shifting, thereby confirming the time delay determined through the cross-correlation.

After the time delay has been determined, it can be compared with empirical data collected from various test subjects and/or calculated data to determine whether the patient under evaluation would or would not benefit from cardiac resynchronization therapy (CRT). In some embodiments, a threshold time delay can be established over which CRT is recommended. Such a threshold can be identified, for example, with reference to negative and positive controls, i.e., healthy subjects and heart disease subjects who have benefited from CRT. In other embodiments, a correlation value derived from the correlation analysis can be used as an aid in making the CRT determination. Such a value can range between +1 to −1, with +1 being perfect correlation and −1 being perfectly opposite correlation.

It is noted that the dyssynchrony determination can be made relative to more than a single pair of opposed points of the myocardial walls. For example, a “global” estimation of dyssynchrony can be determined by calculating time delays between multiple pairs of points and then averaging those time delays. In other embodiments, time delays can be calculated for multiple pairs of points and the maximum time delay can be used in the determination as to whether to prescribe CRT. In still further embodiments, time delays can be calculated for multiple pairs of points and different weights can be assigned to those time delays when considering whether or not to prescribe CRT.

In addition to global estimation of dyssynchrony, “regional” estimation of dyssynchrony can be performed. For example, multiple local profiles can be generated for pairs of points on the myocardial walls, the local profiles averaged to develop a global profile, and then each individual local profile cross-correlated with the global profile to provide an indication of dyssynchrony as to each local region of the heart. Such regional estimation of dyssynchrony may assist a physician in determining where to place a pacing wire of a pacemaker, for example in the region of latest activation.

It is further noted that various different time periods can be considered in generating the heart function profiles. In some embodiments, the time period comprises one complete heart cycle. Alternatively, the time period can comprise two or more contiguous, complete heart cycles. In still other embodiments, the time period comprises one systole or diastole phase, multiple systole phases, or multiple diastole phases. When multiple systole or diastole phases are considered, profiles for each phase can, for example, be averaged with each other to yield averaged profiles that can be cross-correlated.

Testing was conducted to confirm the benefits of quantifying cardiac dyssynchrony using cross-correlation in the manner described above. The subjects tested and the methods used in the testing are described in the following.

Eleven positive controls were identified retrospectively from a database of patients who have received CRT at Emory University Crawford Long Hospital. Inclusion criteria were: (1) TDI and two-dimensional echocardiogram at baseline and three months after CRT, (2) clinical evaluation at baseline and three months after CRT including six-minute hall walk, quality of life score according to the Minnesota Living with Heart Failure questionnaire, and New York Heart Association (NYHA) classification, and (3) a positive response to CRT defined as a decrease in left ventricle (LV) end-systolic volume of at least 15% three months after pacemaker implantation. All patients fit standard CRT selection criteria of ejection fraction <35%, QRS duration ≧120 ms, and NYHA class III or IV heart failure. Patients with atrial fibrillation or chronic right ventricle (RV) pacing were not excluded. Patients had a mean age of 68±14 years and eight of the eleven were male. Five patients had ischemic etiology, four had a right-ventricular pacemaker at baseline, and one patient had atrial fibrillation.

Twelve adult volunteers (mean age 29±7 years) with no known history of cardiac disease, a normal two-dimensional echocardiogram, and normal twelve-lead electrocardiogram were identified as negative controls.

Ejection fraction was quantified using the modified Simpson's rule. End-systolic and end-diastolic dimensions were measured from M-mode parasternal short axis mid-papillary images. Mitrel regurgitation was quantified as the average area of the jet on color flow Doppler from apical 2- and 4-chamber views and also as the ratio of the jet to left atrial area in both views.

Apical 2-, 3-, and 4-chamber tissue Doppler images of the myocardium were acquired with a Vivid 7 system (GE Vingmed, Horten, Norway). The myocardial walls were aligned parallel to the Doppler beam to minimize the angle of insonation, and frame rate was optimized from 100 to 140 Hertz (Hz). Pulsed Doppler images of the aortic outflow tract were acquired for post-processing.

All studies were confirmed to be technically adequate by an independent cardiologist. EchoPAC PC post-processing software (version 4.0.3, GE Vingmed, Horten, Norway) was used to export velocity curves from the TDI data. An average velocity curve was generated from three cardiac cycles of velocity data prior to measuring times-to-peak. Pulsed Doppler of the aortic outflow tract was used to define systole. Myocardial velocity data was exported from the twelve basal and mid-wall segments of the LV.

Times-to-peak systolic velocities were automatically identified by a computer program written in MatLab quantitative analysis software (version 7.10, MathWorks, Inc., Natick, Md.). The program imported velocity curves and aortic valve opening and closure from the EchoPAC software and exported the time from the Q-wave to the maximum velocity in the ejection phase. This was done to eliminate any error due to observer bias in selection of peak velocities.

Three published dyssynchrony parameters were calculated from these time-to-peak values: (1) basal septal-to-lateral delay in time-to-peak systolic velocity (SLD) (2) maximum difference in time-to-peak systolic velocity between any two of the basal septal, lateral, anterior, and inferior LV segments (MaxDiff), and (3) the standard deviation of time-to-peak systolic velocity in the twelve basal and mid-wall segments of the LV (Ts-SD).

Three contiguous cycles of velocity data were exported from the basal segments of the six standard LV walls (septum and lateral walls in 4-chamber view, anteroseptal and posterior walls in 3-chamber view, and anterior and inferior walls in 2-chamber view). Velocity curves were imported into MatLab. The normalized cross-correlation spectrum was computed between two velocity curves by shifting one curve in time relative to the other curve and computing the normalized correlation between the curves for each time shift. A normalized correlation value of 1 therefore meant the two curves were perfectly synchronous in time while a value of −1 meant the two curves were completely dyssynchronous. The time shift between the two curves that resulted in the maximum correlation value was defined as the temporal delay between the two curves. This temporal delay was calculated from opposing basal ventricular segments in each apical view (i.e. the temporal delay derived from the cross-correlation spectrum was calculated for the septal versus lateral basal velocity curves, the anterior versus inferior basal velocity curves, and the anteroseptal versus posterior basal velocity curves). Global dyssynchrony was defined as the maximum absolute value of these three temporal delays. This maximum delay is referred to as the cross-correlation delay (XCD).

SLD, MaxDiff, and Ts-SD were calculated with the exact same velocity curves used to calculate XCD. Dyssynchrony parameters were compared with two criteria: (1) ability to discriminate between the positive and negative controls with a numerical threshold and (2) ability to detect the reduction in dyssynchrony due to CRT (quantified three months after implantation).

SPSS version 14.0 (SPSS Inc., Chicago, Ill.) was used to calculate the area under the receiver operating characteristic (ROC) curve as a measure of a parameter's ability to discriminate between the positive and negative controls. Areas under the ROC curve were statistically compared using the method described by Hanley and McNeil. The mean values of each parameter in the positive and negative controls were compared with an unpaired t-test. Baseline and three-month post-CRT values of dyssynchrony were compared for each parameter using a paired t-test. A value of p<0.05 was defined as statistically significant. XCD was quantified twice by the same observer and once by an independent observer in all subjects in order to quantify intra and inter-observer reproducibility.

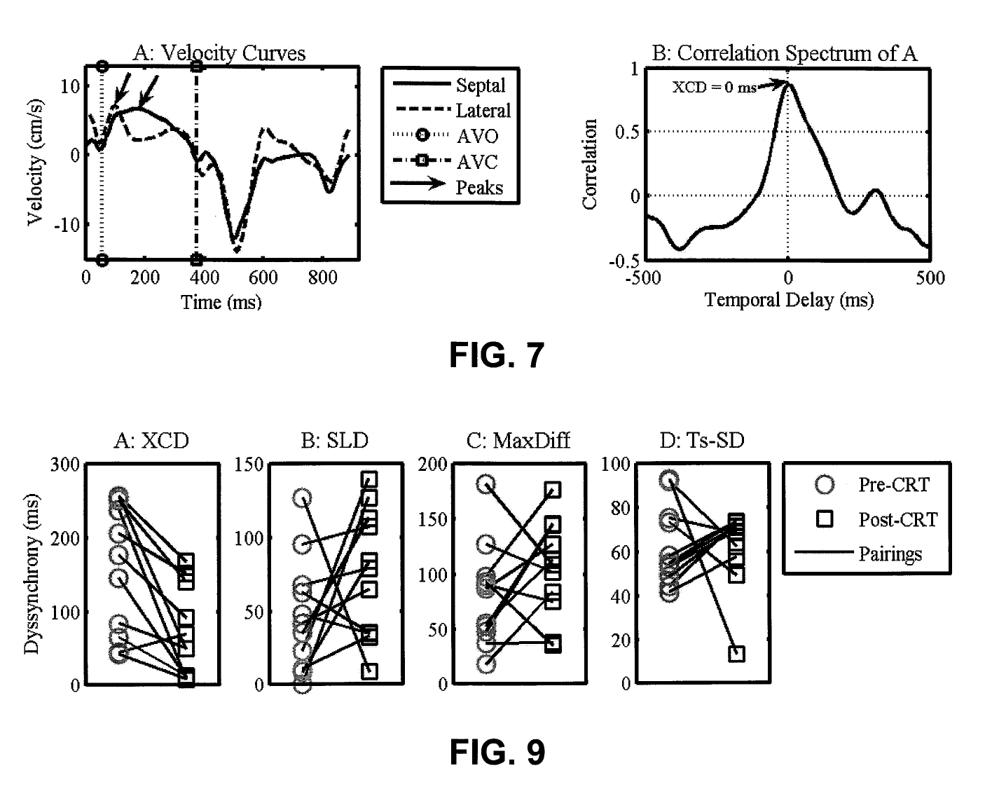

Mean inter- and intra-observer reproducibility in the negative controls was 0.3±0.8% and 0.5±1%, respectively. Table 1 shows the mean dyssynchrony and percentage of negative controls exhibiting dyssynchrony according to each parameter. SLD, MaxDiff, and Ts-SD showed dyssynchrony in six, seven, and six out of the twelve negative controls, respectively. XCD showed dyssynchrony in zero subjects. FIG. 7 shows an example of a representative negative control who exhibited dyssynchrony according to all parameters except XCD.

TABLE 1

| | Value in neg. | Dyssynchrony | Neg. controls with | | |
|---|---|---|---|---|---|
| Parameter | control group, ms | threshold, ms | Dyssynchroney | Sensitivity | Specificity |
| XCD | 9 ± 5 | 31 | 0% | 100% | 100% |
| SLD | 53 ± 44 | 60 | 50% | 36% | 50% |
| MaxDiff | 56 ± 43 | 65 | 58% | 55% | 42% |
| Ts-SD | 28 ± 16 | 34.4 | 50% | 100% | 50% |

Mean inter and intra-observer reproducibility in the positive controls was 6.1±8.7% and 5.9±8.8%, respectively. Table 2 reports the mean dyssynchrony, echocardiographic, and clinical characteristics of the positive control group both before and three months after CRT. Patients showed a significant (p<0.05) decline in LV end-systolic volume, LV end-diastolic volume, and NYHA functional class. Patients also showed a significant (p<0.05) increase in LV ejection fraction and quality of life. The mean mitral regurgitation decreased, but the decline was not significant. Similarly, the mean six-minute hall walk distance increased but was not significant.

TABLE 2

| Variable | Pre-CRT | Post-CRT | P value |
|---|---|---|---|
| Dyssynchrony parameters | | | |
| XCD, ms | 160 ± 88 | 69 ± 61 | .003 |
| SLD, ms | 47 ± 39 | 75 ± 44 | .220 |
| MaxDiff, ms | 81 ± 46 | 104 ± 44 | .220 |
| Ts-SD, ms | 63 ± 18 | 62 ± 18 | .953 |
| Enchocardiographic measurements | | | |
| LV end-systolic volume, mL | 145 ± 76 | 88 ± 38 | .002 |
| LV end-diastolic volume, mL | 188 ± 84 | 139 ± 47 | .004 |
| LV end-systolic dimension, cm | 5.9 ± 1.1 | 4.9 ± 1.7 | .01 |
| Lv end-diastolic dimension, cm | 6.6 ± 1.1 | 6.0 ± 1.7 | .161 |
| LV ejection fraction, % | 24 ± 7 | 37 ± 10 | <.001 |
| Mitral regurgitation area, cm² | 7.4 ± 4.5 | 5.2 ± 3.8 | .082 |
| Ratio of mitral regurgitation | 34 ± 17 | 23 ± 15 | .06 |
| area to left atrial area, % | | | |
| QRS duration, ms | 175 ± 23 | 160 ± 27 | .18 |
| NYHA class | 3.1 ± 0.3 | 1.8 ± 0.8 | <.001 |
| Quality-of-life score | 54 ± 27 | 26 ± 29 | .007 |
| 6-min Hall walk, m | 227 ± 127 | 327 ± 103 | .005 |

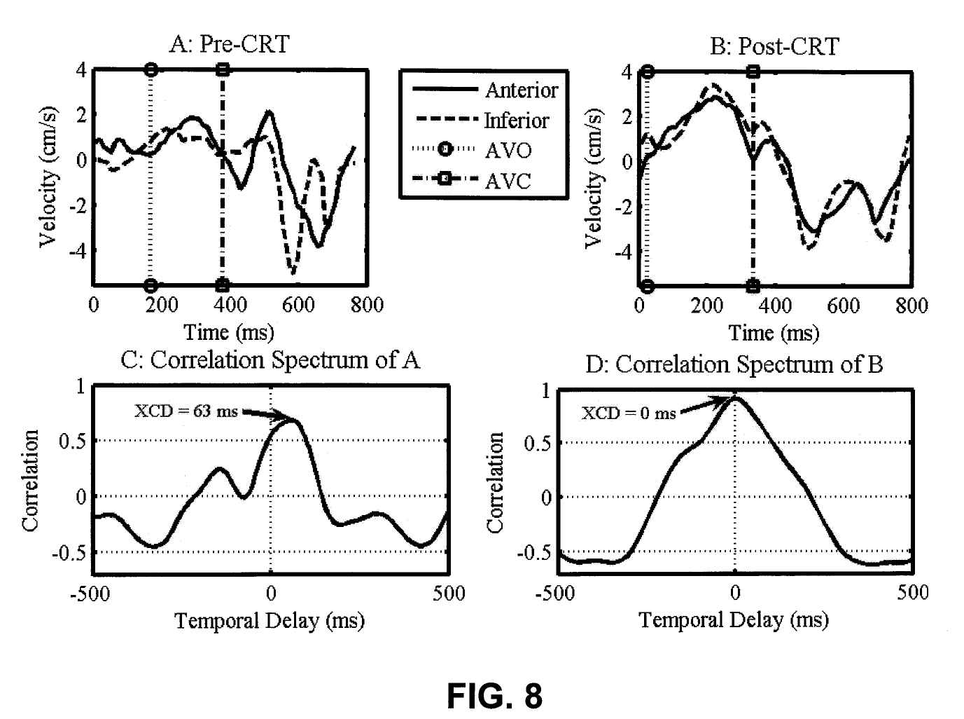

XCD was the only dyssynchrony parameter that showed a significant reduction (57%) from baseline to three months after biventricular pacemaker implantation. Ten of the eleven positive controls showed a decrease in XCD three months after CRT compared with only three, four, and four for SLD, MaxDiff, and Ts-SD, respectively. FIG. 8 shows an example of a representative positive control who exhibited a decrease in XCD following CRT. Figure three shows values of each dyssynchrony parameter for all the positive controls (both before and three months after CRT). Four, six, and eleven of the total eleven positive controls showed dyssynchrony according to published threshold values (reported in Table 1) for SLD, MaxDiff, and Ts-SD, respectively.

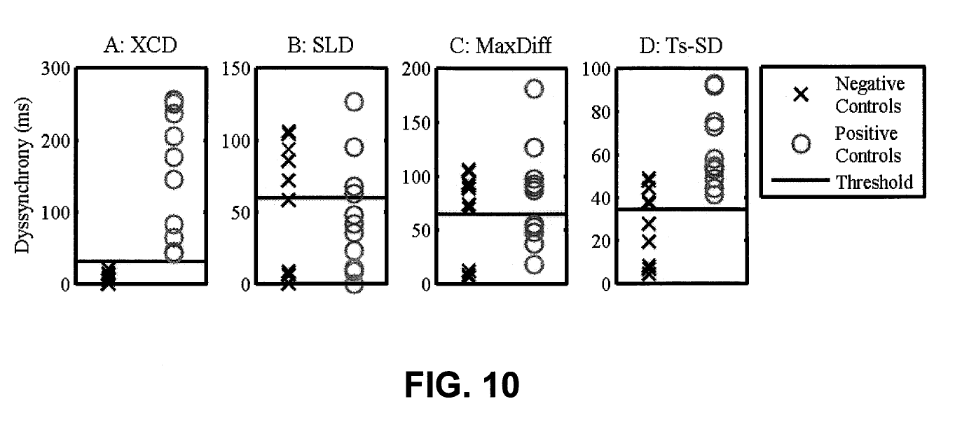

XCD and Ts-SD were significantly different in the positive and negative controls (p<0.0001, p=0.0001, respectively). SLD and MaxDiff were not significantly different between the positive and negative control groups (p=0.754 and p=0.195, respectively). FIG. 10 shows values of each dyssynchrony parameter for all the positive and negative controls along with threshold values used to diagnose dyssynchrony.

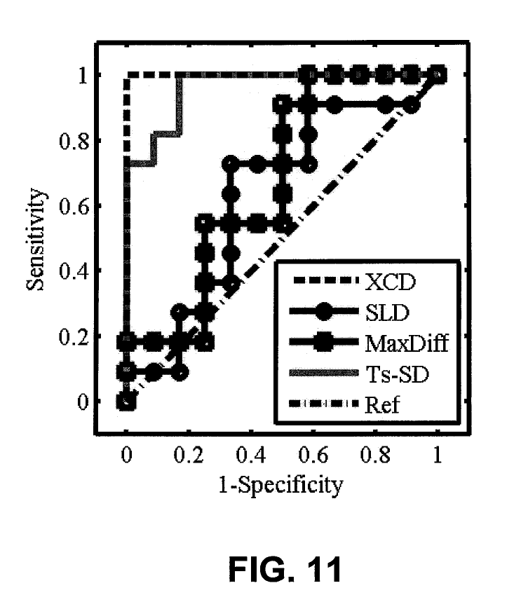

FIG. 11 shows the ROC comparison of the dyssynchrony parameters. XCD and Ts-SD were the only parameters which demonstrated significant discrimination between positive and negative controls (both p<0.0001). XCD and Ts-SD both had areas under the ROC curve that were significantly higher than those of SLD and MaxDiff (p<0.01 for all four comparisons) (FIG. 11). ROC analysis determined that a threshold XCD of 31 ms had the highest sensitivity and specificity of all dyssynchrony parameters (100 and 100%, respectively) (Table 1).

As can be appreciated from the above, the systems and methods of the present disclosure can be used to quantify dyssynchrony with higher accuracy than current techniques. Instead of comparing a single point of each cardiac function (e.g., velocity) profile, tens or hundreds of points of the profiles for a given time period of heart function can be evaluated and taken into consideration.

We claim:

1. A method for quantifying cardiac dyssynchrony, comprising:
capturing images of the heart over time as it beats;
generating heart function profiles for discrete portions of the myocardium from the captured images, the heart function profiles each pertaining to a heart function parameter as a function of time; and
performing cross-correlation on the generated heart function profiles to yield a cross-correlation function that identifies a time delay at which the heart function profiles temporally correlate most closely, the time delay being indicative of a degree of cardiac dyssynchrony.

2. The method of claim 1, wherein capturing images comprises capturing images using tissue Doppler velocity imaging.

3. The method of claim 1, wherein the heart function profiles are generated in relation to image data that spans one or more complete cardiac cycles.

4. The method of claim 1, wherein the heart function profiles are generated in relation to image data that spans one or more systole periods.

5. The method of claim 1, wherein the heart function profiles are generated in relation to image data that spans one or more diastole periods.

6. The method of claim 1, wherein generating heart function profiles comprises generating heart function profiles for a first point on a first myocardium wall and a second point on a second myocardium wall opposite the first myocardium wall.

7. The method of claim 1, wherein generating heart function profiles comprises generating heart function profiles for two points on opposing walls of the left ventricle.

8. The method of claim 1, wherein generating heart function profiles comprises generating one of velocity profiles, displacement profiles, strain rate profiles, or strain profiles.

9. A method for quantifying cardiac dyssynchrony, comprising:
generating a first velocity profile for a first point on a first myocardium wall, the first velocity profile being indicative of the velocity of the first point as a function of time;
generating a second velocity profile for a second point on a second myocardium wall opposite the first myocardium wall, the second velocity profile being indicative of the velocity of the second point as a function of time;
cross-correlating the first and second velocity profiles to yield a cross-correlation function whose maximum identifies a time delay at which the velocity profiles temporally correlate most closely; and
identifying the time delay, the time delay being indicative of a degree of cardiac dyssynchrony.

10. The method of claim 9, wherein the first and second points are points on opposing walls of the left ventricle.

11. The method of claim 9, further comprising generating further velocity profiles for other points on the myocardium walls and cross-correlating pairs of the further velocity profiles that pertain to opposing points on the myocardium walls to generate multiple time delays.

12. The method of claim 11, further comprising averaging the multiple time delays and using the averaged time delay to quantify cardiac dyssynchrony.

13. A computer-readable medium that stores a system comprising:
logic configured to receive heart function profiles that pertain to a heart function parameter as a function of time; and
logic configured to perform cross-correlation on the heart function profiles to yield a cross-correlation function that identifies a time delay at which the heart function profiles temporally correlate most closely, the time delay being indicative of a degree of cardiac dyssynchrony.

14. The computer-readable medium of claim 13, wherein the logic configured to receive heart function profiles is configured to receive a first heart function profile for a first point of a first myocardium wall and a second heart function profile for a second point of a second myocardium wall opposite the first myocardium wall.

15. The computer-readable medium of claim 13, wherein the logic configured to receive heart function profiles is configured to receive heart function profiles that spans one or more complete cardiac cycles.

16. The computer-readable medium of claim 13, wherein the logic configured to receive heart function profiles is configured to receive heart function profiles that only spans one or more systole periods.

17. The computer-readable medium of claim 13, wherein the logic configured to receive heart function profiles is configured to receive heart function profiles that only spans one or more diastole periods.

18. The computer-readable medium of claim 13, wherein the logic configured to receive heart function profiles is configured to receive heart function profiles that pertain to points on opposing walls of the left ventricle.

19. The computer-readable medium of claim 13, wherein the logic configured to receive heart function profiles is configured to receive velocity profiles that pertain to velocities of discrete portion of the myocardium as functions of time.

20. A system comprising:
an imaging system configured to capture images of the heart during a designated period of time during which the heart is beating;
a profile generation system configured to receive image data captured by the imaging system, track discrete points of the myocardium over time across the image data, and generate separate heart function profiles for each of the discrete points, the heart function profiles each pertaining to a heart function parameter as a function of time; and
a cardiac dyssynchrony quantification system configured to receive the heart function profiles generated by the profile generation system and cross-correlate the heart function profiles to determine a delay time at which the heart function profiles temporally correlate most closely, the time delay being indicative of a degree of cardiac dyssynchrony.

21. The system of claim 20, wherein the imaging system is a tissue Doppler imaging system.

22. The system of claim 20, wherein the profile generation system is configured to generate velocity profiles for each of the discrete points.

23. The system of claim 20, wherein the profile generation system is configured to generate heart function profiles for one or more complete cardiac cycles.

24. The system of claim 20, wherein the profile generation system is configured to generate heart function profiles only for one or more systole periods.

25. The system of claim 20, wherein the profile generation system is configured to generate heart function profiles only for one or more diastole periods.
